(19) United States
(12) Patent Application Publication
(10) Pub. No.: US 2006/0052658 A1
(43) Pub. Date: Mar. 9, 2006

(54) Clothing having an electromagnetic effect

(76) Inventor: Sabetay Ozpapu, Istanbul (TR)

Correspondence Address: DENNISON, SCHULTZ, DOUGHERTY & MACDONALD, 1727 KING STREET, SUITE 105, ALEXANDRIA, VA, 22314, US

(21) Appl. No.: 10/531,879

(22) PCT Filed: Mar. 5, 2004
(86) PCT No.: PCT/IB04/50205

§ 371 Date: Aug. 3, 2005

(30) Foreign Application Priority Data
Mar. 19, 2003 (TR) 2003/00357

(51) Int. Cl. A61N 1/00

(52) U.S. Cl. 600/15

(57) ABSTRACT

Clothing used in the treatment of the pain through the effects of electric and magnetic fields. In a preferred embodiment of the invention, the clothing includes a band having a conductive wire and two magnets at the ends of the band.

This invention is related to clothing for pain treatment through the effects of the electric and magnetic fields.

As it is known, a current occurs as a reaction to the change in the conductors in a magnetic field where the flow is changed (Lentz Law). Furthermore, a difference in tension occurs in the conductors which are subjected to a magnetic field and accommodate an electric current due to the collection of the electrons to one side according to the magnetic current (Hall effect). Similarly the motions of the loaded particles change in the conductors in the electric fields.

The organisms including human beings which consist of water comprising 75% of melted salts are substantially complicated electrolyte systems. In these organisms, liquids like blood plasma are in close connection with the cells and the nerve cells which are called as neurons. Similarly, cytoplasm in neurons comprises dissolved potassium chloride (KCl). The solution gains the prospect of electrical conductivity due to the dissolved K⁺ and Cl⁻ ions. In case a tension V (Volt) is applied to a solution generally called as electrolyte, it will put up a definite amount of resistance R (Ohm) against the electric current I (Ampere) (Ohm Law). This results in a temperature in direct proportion with the square of the current and the resistance in the conductor. The negative loaded ions which take the place of electrons in the metals provide the electrolyte with above mentioned prospects under the effect of the magnetic and electric field.

The effects of the magnetic and electric fields described above on the organisms are known for several years. In this purpose various medicaments, pain relieving methods known as magnetotherapy and electrotherapy and related means have been developed.

In the state of art, U.S. Pat. No. 4,480,596 defines an elastic belt which exposes the waist part of the body to the magnetic current. In this invention, many a temporary magnets which can be fixed and removed from the belt are utilized as the source of magnetism.

In the prior art, CH1184616 discloses a clothing without sleeves comprising two layer of leather and magnetic particles.

GB2377179 describes a wristband at two open ends of which temporary magnets are attached. In this invention, a metal plate which is made preferably of stainless steel and does not have a magnetic prospect is probably placed in the housings where the magnets are attached. The poles of the magnets are opposite to each other.

A pant comprising 24 bio-magnets is shown in URL address http://www.buyamag.com/cpi-bin/html/panties.htm.

U.S. Pat. No. 4,765,310 as another embodiment discloses a device used in the treatment by means of the common effect of a magnetic field made of a magnet and an electric field connected to the difference in tension caused by a magnet on a coil.

GB2368287 defines a long nucleus which can be induced magnetically and a device comprising an insulated conductive wire wrapped around the said nucleus. In this invention the said wire grabs the electric current transmitted from the body and induces a magnetic field which is utilized for the treatment of the body. This invention can be added to clothing. Furthermore, the nucleus material can be weaved as a grid and used as an elastic band.

In the conventional applications, many complicated embodiments have been used. In addition these embodiments utilized from the electric field and the magnetic field separately, and in those which utilized the both many difficulties were encountered.

The object of this invention is to realize clothing easy to produce and purchase having a simple structure for the fast and efficient pain treatment using both the magnetic and electric field.

The clothing realized in order to attain above mentioned objects of the present invention has been illustrated in the attached drawings, wherein;

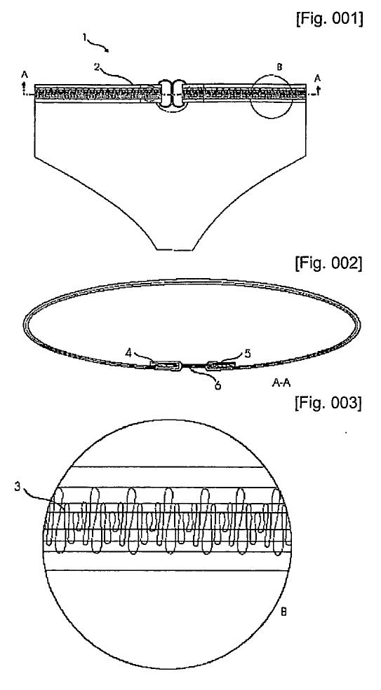

FIG. 1 is a cross view of the clothing,

FIG. 2 is a detailed view of the clothing,

FIG. 3 is a cross section view of the clothing.

The components shown in the figures have been enumerated as below;

1. Clothing

2. Band

3. Wire

4. Pocket

5. Magnet

6. Holder

The clothing (1) which is the subject of this invention for the pain treatment can be produced as trousers, pants, skirt and etc.

There is a wire (3) or a band (2) comprising a strip in the waist part of the clothing (1). The said band is preferably elastic. In order to place the wire on the band, a wire can be weaved together with another type of thread (FIG. 3) or the wire can be attached by means of pasting on the fabric or any other methods. The conductive wire (3) can extend along the total length of the band (FIG. 2) or may not extent to some extent. If the conductive wire (3) extends along the total length of the band, an edge of the band should be left open to prevent short circuit. In order to avoid the contact of the wire with the skin, the band can be covered by an insulated material or the distance between the wire and the skin may be increased via a thread thicker than the wire.

The wire used in every band is produced preferably from a ductile metal which has a high conductivity coefficient like copper and gold. The zigzag or sinusoidal, wavy application of the wire instead of a straight way prevents the wire from being broken during the movements of the body. In another embodiment of the invention the wire can be used as serial or parallel connection.

In any case, preferably two magnets (5) are placed in the open ends of the band (2) or the part lacking the wire. These magnets (5) are preferably attached to the pockets (4). These pockets (4) are formed by being folded and sewed from the ends of the band (FIG. 2). On the other hand it may be formed by adding another fabric which enables the formation of more than one pocket and replace or various use of the magnets on the clothing. The magnets should be placed according to the body part where the pain will be treated. For example, in order to relieve the menstruation pains, magnets are placed to the front part of the body and especially very close to the ovular. When the back pains are treated, the said magnets are placed on the back part of the body.

Magnets (5) having a thickness of 0.5-13000 and preferably 12500 Gauss are preferred. It is important that the thickness of the magnets is 0.05-10 mm. The magnets can be in a circle, square, triangle, etc. shape. A magnet in a metal nucleus form induced by an electric source can also be used. The poles of the magnets (polarizations) are preferably the same, but may differ as well.

If two ends of the band are used open, these said ends are hold together by means of a holder (6) passing through the pockets (FIG. 1 and 2). Since the distance between two ends of the holder or the band determines the current and the effect field of the magnets, the said distance should be determined according to the intensity of the pains in the body. In a preferred embodiment of the invention, the said distance is 0.5-50 mm.

In another embodiment of the invention, the said holder (6) is formed in a way that its width can be adjusted like a belt to allow the adjustment of the band length and distance according to the intensity of the pains and sizes of the users.

In a preferred embodiment of the invention, the clothing is made of a cotton fabric.

In another embodiment of the invention, the clothing is produced as underpants and of disposable material for the disposal of the product easily by the user. The said disposable material can be based on celluloses and/or plastic. Furthermore a hygienic pad can be added to the crotch of the product.

In a preferred embodiment of the invention, the clothing is in a form of underpants made of cotton fabric. The band is produced as rubber by wavy weaving of a synthetic thread with a thin wire. Afterwards the ends are folded and sewed to form pockets. Each magnet is placed in these pockets with their poles in the same direction and two ends of the band are joined by means of the holder through the pockets. The band is sewed to the underpants in a way that the magnets face with the front part.

The clothing which is the subject of the invention provides the treatment of various pains and especially the pains in ovular during the menstruation period. The user who wears the clothing which is the subject of the invention has a magnetic field formed by the magnets in the pockets efficient on the ovular area. This area affects the movements of the potassium chloride ions in the neurons and blood plasma in the ovular. Furthermore a current arises in the conductive copper wire induced by the magnets and the said current contributes to the formation of a magnetic and electric field. This double effect can be beneficial in the treatment of the pains. It has observed in the users having severe menstruation pains that the pains are relieved in 45-60 minutes.

The clothing which is the subject of the invention is realized to be efficient in the treatment or reduction of the pains through the effects of the electric and magnetic field. The production of this clothing is easy and cost saving.

1. Clothing used in the treatment and reduction of the pains characterized with a band (2), one or more conductive wire (3) which can be fixed onto a band and two or more magnets (5) fixed onto the said band (2).

2. Clothing as defined in claim 1 characterized with a band (2) on the waist part.

3. Clothing as defined in claim 1 characterized with an elastic band (2).

4. Clothing as defined in claim 1 characterized with a wire (3) having the thickness of a thread.

5. Clothing as defined in claim 1 characterized with a wire (3) having the width of a strip.

6. Clothing as defined in claim 1 characterized with a wire (3) which is 9 weaved with another thread in the band.

7. Clothing as defined in claim 6 characterized with a band made of a thread having a thickness more than the wire.

8. Clothing as defined in claim 1 characterized with a wire (3) which is adhered on the band.

9. Clothing as defined in claim 1 characterized with a band comprising a wire (3) extending along the entire length.

10. Clothing as defined in claim 1 characterized with a band comprising a wire extending to an extent.

11. Clothing as defined in claim 1 characterized with a wire covered by an insulation material.

12. Clothing as defined in claim 1 characterized with a band having an open end.

13. Clothing as defined in claim 1 characterized with a wire made of a ductile metal having a high coefficiency.

14. Clothing as defined in claim 1 characterized with a wire attached to the band in a zigzag way.

15. Clothing as defined in claim 1 characterized with a wire attached to the band in a wavy way.

16. Clothing as defined in claim 1 characterized with wires having serial connections.

17. Clothing as defined in claim 1 characterized with wires having parallel connections.

18. Clothing as defined in claim 1 comprising two or more pockets (4) for the placement of the magnets (5).

19. Clothing as defined in claim 18 characterized with a pocket formed by being folded and sewed from the ends of the band.

20. Clothing as defined in claim 18 characterized with a pocket formed by the addition of another fabric on the band.

21-32. (canceled)
